(19) United States
(12) Patent Application Publication
(10) Pub. No.: US 2026/0158212 A1
(43) Pub. Date: Jun. 11, 2026

(54) PERISTALTIC PUMP FOR REUSABLE MODULE IN WEARABLE FLUID DELIVERY DEVICE

(71) Applicant: Becton, Dickinson and Company, Franklin Lakes, NJ (US)

(72) Inventors: Vincent Delobelle, Domene (FR); Clementine Le Loc'h, Meylan (FR); Nicolas Besson, Treffort (FR)

(73) Assignee: Becton, Dickinson and Company, Franklin Lakes, NJ (US)

(21) Appl. No.: 19/408,435

(22) Filed: Dec. 4, 2025

Related U.S. Application Data
(60) Provisional application No. 63/729,682, filed on Dec. 9, 2024.

(51) Int. Cl. A61M 5/142

(52) U.S. Cl. CPC A61M 5/14228; A61M 5/14248; A61M 2005/14268; A61M 2205/3331

(57) ABSTRACT

A wearable fluid delivery device (FDD) has a fluidic module and an electromechanical module. The fluidic module has a fluid path from a fluid reservoir to an outlet to a patient that is separate from the electromechanical module and its components that control the delivery of the fluid to the patient. The FDD can have a releasably coupled first housing and second housing that enclose a respective one of the fluidic module and the electromechanical module. The first and second housings when connected have respective ones of aligned fluid path access opening(s) and pump access opening(s) to allow a peristaltic-type pump in the electromechanical module in the second housing to controllably contact a compressible portion of the fluid path exposed externally with respect to first housing via the fluid path access opening(s).

This application claims the benefit of U.S. Provisional Application Ser. No. 63/729,682, filed Dec. 9, 2024, the entire contents of which are incorporated herein by reference.

### BACKGROUND

### Field

The present disclosure relates generally to wearable fluid delivery devices for delivering a medicament to a patient. The present disclosure particularly relates to a fluid delivery device having a fluidics module with a fluid path from a fluid reservoir to an outlet to the patient that is separate and detachable from an electromechanical module with components to control the delivery of the fluid from the fluid reservoir to the patient. Further, the present disclosure relates to the fluid delivery device having a peristaltic pump in the electromechanical module that is external to the fluid path that controllably contacts the fluid path to cause fluid therein to be transported (e.g., to move fluid from the fluid reservoir to the outlet via the fluid path).

### Description of Related Art

Bolus and/or infusion pump therapy generally requires an infusion cannula, typically in the form of an infusion needle and/or a flexible catheter, that pierces the patient's skin and through which infusion of a medicament takes place. Infusion pump therapy offers the advantages of continuous infusion, precision dosing, and programmable delivery schedules.

To facilitate drug or medicament delivery therapy, there are generally two types of pumps, namely, conventional pumps and patch pumps. Conventional pumps require the use of a disposable component, typically referred to as an infusion set, tubing set or pump set, which conveys a medicament from a reservoir within the pump into the skin of the user. The infusion set consists of a pump connector, a length of tubing, and a hub or base from which a cannula in the form of a hollow metal infusion needle or flexible plastic catheter extends. The base typically has an adhesive that retains the base on the skin surface of a user during use of the pump. The cannula can be inserted onto the skin manually or with the aid of a manual or automatic insertion device. The insertion device may be a separate unit required by the user.

Unlike a conventional infusion pump and infusion set combination, a patch pump is an integrated device that combines most or all of the fluidic components, including the fluid reservoir, pumping mechanism and mechanism for inserting the cannula, in a single housing that is adhesively attached to an infusion site on the patient's skin, and does not require the use of a separate infusion or tubing set. A patch pump containing a medicament adheres to the patient's skin and delivers the medicament over a period of time or at a selected time via an integrated subcutaneous cannula. Some patch pumps may wirelessly communicate with a separate controller device, while others are completely self-contained. Such devices can be replaced on a frequent basis, such as every three days, when the medicament reservoir is exhausted or a component malfunctions, or when complications may otherwise occur, such as restriction in the cannula or the infusion site or other occlusion.

### SUMMARY

In accordance with illustrative embodiments of the present disclosure, a wearable fluid delivery device (FDD) comprises two parts; that is, a fluidic module and an electromechanical module. The fluidics module has a fluid path from a fluid reservoir to an outlet to the patient that is separate and detachable from the electromechanical module and its components that control the delivery of the fluid from the fluid reservoir to the patient. The electromechanical module can have a pump that is external to the fluid path in the fluidic module and that controllably cooperates with the fluid path to move fluid in the fluid path (e.g., move fluid that has been provided to the fluid path from the fluid reservoir to the outlet to the patient). The wearable FDD realizes a number of advantages described below.

It is an aspect of illustrative embodiments to provide a wearable fluid delivery device comprising: a first housing that defines an interior comprising a fluidics module and that has a fluid path access opening to an exterior of the first housing, the fluidics module comprising a reservoir of fluid and a fluid path that fluidically connects the reservoir to an outlet port configured to output the fluid from the fluid path to a patient, the fluid path comprising a compressible section thereof that extends from the interior of the first housing through the fluid path access opening to an exterior of the first housing; and a second housing separate from the first housing and configured to be releasably connected to the first housing and operated as the wearable fluid delivery device whereby the connected first housing and second housing are dimensioned for detachable application at an injection site on a patient's skin. The second housing comprises an electromechanical module and a pump access opening. The electromechanical module comprises a pump connected to a motor and electronics configured to operate the motor. The fluid path access opening and the pump access opening are at least partially aligned when the second housing is connected to first housing. The pump comprises a rotor connected to at least one pump head contact member disposed at the pump access opening of the second housing. The motor is controlled by the electronics to selectively contact the compressible section of the fluid path with the at least one pump head contact member for peristaltic pumping operations chosen from filling the fluid path with fluid from the reservoir and transporting the fluid in the fluid path toward the outlet port.

In accordance with aspects of illustrative embodiments, the first housing is disposable after a first use thereof and replaceable by new first housing that is similar to the used first housing, and the second housing is reusable by disconnecting the used first housing from the second housing and attaching the new first housing to the second housing to engage the at least one pump head contact member with the compressible section of the fluid path in the new first housing.

In accordance with aspects of illustrative embodiments, at least one of the connected first housing and second housing comprises an adhesive patch to attach the connected first housing and second housing to the patient's skin.

In accordance with aspects of illustrative embodiments, the compressible section of the fluid path is chosen from tubing and hose.

In accordance with aspects of illustrative embodiments, each of the first housing and the second housing has a main portion that is characterized by a respective main housing thickness, and the connected first housing and second housing assembly has a connected housing thickness that is the same as the main housing thickness.

In accordance with aspects of illustrative embodiments, the first housing has an overlapping portion that comprises the compressible section of the fluid path and that is combined with an overlapping portion of the second housing to connect the pump of the second housing to the compressible section of the fluid path.

In accordance with aspects of illustrative embodiments, a section thickness of the combined overlapping portions does not exceed the main housing thickness.

In accordance with aspects of illustrative embodiments, the overlapping portion of the first housing is moved laterally with respect to the overlapping portion of the second housing to connect the first housing to the second housing, the overlapping portion of the first housing having at least part of the compressible section of the fluid path exposed along a side wall of the first housing and disposed between the side wall of the first housing and an adjacent side wall of the second housing.

In accordance with aspects of illustrative embodiments, the side wall of the first housing comprises a recess with a fluid path access opening arranged therein to expose the at least part compressible section of the fluid path.

In accordance with aspects of illustrative embodiments, the at least one pump head contact member extends from the second housing to contact the exposed compressible section of the fluid path within the recess when the first housing and the second housing are connected.

In accordance with aspects of illustrative embodiments, the overlapping portion of the first housing is moved in a vertically stacked arrangement with respect to the overlapping section of the second housing to connect the first housing to the second housing.

In accordance with aspects of illustrative embodiments, the overlapping portion of the first housing is a side wall extension with exposed fluid path tubing arranged along an interior side wall thereof, the side wall extension being dimensioned for placement over a pump head of the pump having at least one pump head contact member in the overlapping portion of the second housing, the side wall extension and the overlapping portion of the second housing being arranged together such that the at least one pump head contact member controllably compresses the tubing against the interior side wall.

In accordance with aspects of illustrative embodiments, the pump is a linear peristaltic pump comprising a spring loaded plate arranged within the second housing, and the compressible section of the fluid path that is exposed at the exterior of the first housing is arranged along the spring loaded plate when the first and second housing are engaged.

In accordance with aspects of illustrative embodiments, the second housing comprises a fluid path access opening from an exterior of the second housing to an interior thereof to receive the compressible section of the fluid path for arrangement along the spring loaded plate.

In accordance with aspects of illustrative embodiments, the wearable fluid delivery device further comprises a retractable member for the pump that controllably extends and retracts the pump head contact member with respect to the compressible section of the fluid path.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims.

### BRIEF DESCRIPTION OF DRAWINGS

The above and/or other aspects and advantages of the illustrative embodiments will be more readily appreciated from the following detailed description, taken in conjunction with the accompanying drawings, of which:

FIG. 1 is an example wearable FDD housing;

FIG. 2 depicts a block diagram of an example embodiment of a fluid delivery device having releasably connected housing parts containing a fluidic module and an electromechanical module respectively, and a pump deployed in the electromechanical module that is configured to cooperate with the fluid path in the fluidic module;

FIGS. 3A, 3B and 3C are diagrams of operations of an example peristaltic pump that can be implemented in a fluid delivery device having releasably connected housing parts in accordance with an example embodiment;

FIG. 3D is an exploded diagram of an example peristaltic pump comprising rollers that can be implemented in a fluid delivery device configured in accordance with an example embodiment;

FIGS. 4A, 4B and 4C are diagrams of an embodiment of a fluid delivery device according to FIG. 2 wherein the releasably connected housing parts containing the fluidic module and the electromechanical module, respectively, are engaged side-by-side and the housing parts overlap at least a small amount when engaged;

FIGS. 5A, 5B, 5C and 5D are diagrams of another embodiment of fluid delivery device according to FIG. 2 wherein the releasably connected housing parts containing the fluidic module and the electromechanical module, respectively, are engaged in a vertically stacked arrangement and the housing parts have overlapping portions when engaged; and

FIGS. 6A, 6B, 6C, 6D, 6E and 6F are diagrams of yet another embodiment of fluid delivery device according to FIG. 2 wherein the releasably connected housing parts containing the fluidic module and the electromechanical module, respectively, are engaged side-by-side and the housing parts do not overlap.

Throughout the drawing figures, like reference numbers will be understood to refer to like elements, features and structures.

### DETAILED DESCRIPTION OF ILLUSTRATIVE EMBODIMENTS

Reference will now be made in detail to illustrative embodiments, which are depicted in the accompanying drawings. The embodiments described herein exemplify, but do not limit, the illustrative embodiments by referring to the drawings.

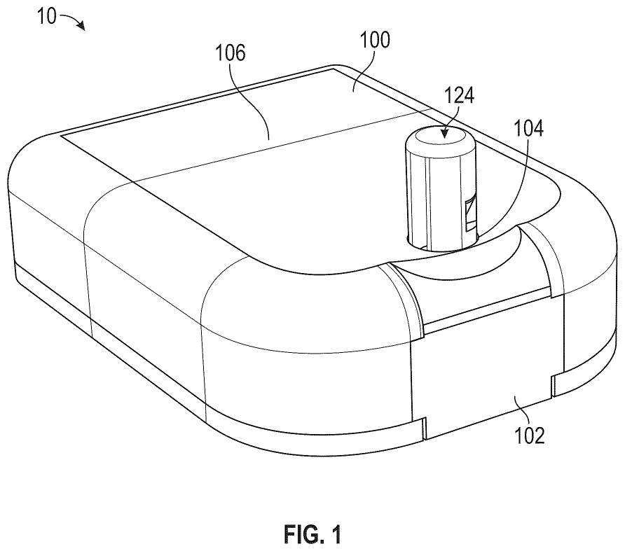

FIG. 1 is an example wearable FDD 10 having a housing 100. In accordance with example embodiments, the housing 100 can be divided into two releasably connected housing parts 102 and 106 comprising the single use fluidic module and the electromechanical module, respectively. FIG. 1 indicates a division of the housing 100 into releasably connected, side by side, housing parts 102 and 106; however, the two releasably connected housing parts 102 and 106 can be instead configured for arrangement as top and bottom releasably connected parts. The two releasably connected housing parts 102 and 106 can be equal or unequal in terms of dimensions, and can have partially overlapping portions thereof. Further, the two releasably connected housing parts 102 and 106 can employ different configurations of components and fluid pathways, and various electrical and mechanical interfaces or connections between their respective fluidic module and electromechanical module, such as described herein with respect to example embodiments shown in the drawing figures.

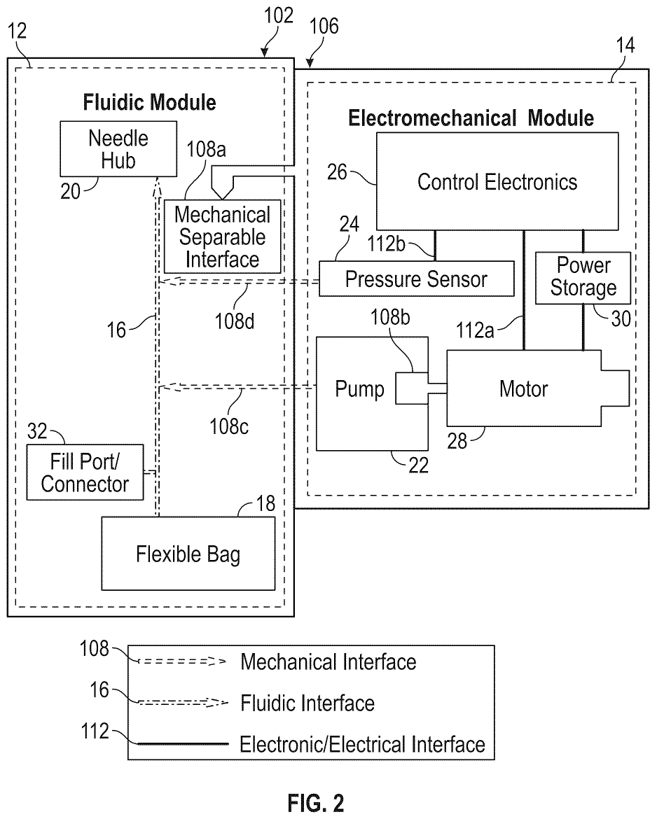

With reference to FIG. 2 and in accordance with illustrative embodiments of the present disclosure, a wearable FDD 10 comprises two parts; that is, a fluidic module 12 and an electromechanical module 14 which can be arranged in the two releasably connected parts 102 and 106, respectively, of the housing 100. The fluidic module 12 components comprise a fluid path indicated at 16 from a fluid reservoir 18 to the patient via an outlet port 20. The outlet port 20 can be, for example, a needle in a needle hub that is fluidically connected to the fluid path 16. The fluid path 16 is separate from the electromechanical module 14 and its components (e.g., a pump 22, a motor 28, control electronics 26, and a power storage subsystem 30 for the FDD that can include batteries) that control the delivery of the fluid from the fluid reservoir 18 to the patient. The housing 106 can have one or more activation buttons (not shown) that when activated (e.g., depressed by a user) cause control electronics to wake-up the FDD 10 and commence a delivery mode as programmed. The programmed delivery mode can be delayed or immediate fluid discharge by pump, pulsed or continuous delivery, among other delivery modes.

With continued reference to FIG. 2, the wearable FDD 10 can have an optional pressure sensor 24 that is external to the fluid path 16. The control electronics 26 are programmed or otherwise configured to receive data from the pressure sensor 24 and determine fluidic pressure in at least a portion of the fluid path 16. The pressure sensor 24 can be provided in either of the fluidic module 12 and the electromechanical module 14. The pump 22 can be, for example, a peristaltic pump that is external to the fluid path 16. The fluid path 16 can be implemented in a number of ways and can have different portions thereof made of different materials such as plastic tubing, and embedded channels in a plate, among other materials and configurations. The reservoir 18 can be a flexible bag reservoir as described in commonly-owned published PCT application WO 2017/053284 and incorporated herein by reference in its entirety. The outlet port 20 can be a catheter and/or needle that is indicated as a needle hub 20, and that can be inserted into patient's skin by an insertion mechanism 124 shown in FIG. 1. The insertion mechanism 124 can be configured, for example, as described in commonly-owned published PCT application WO 2015/164653 and incorporated herein by reference in its entirety. The example embodiment in FIG. 2 has an optional fluid connecter 32 configured as a fill port to the reservoir 18. An example fill port 32 is also described in the above-referenced commonly-owned published PCT application WO 2017/053284 and incorporated herein by reference in its entirety. It is to be understood that other configurations of a reservoir 18, pump 22, outlet port 20 and any associated insertion mechanism 124, and fill port or fluid connector 32 can be used in a FDD 10 in addition to the configurations shown and described in the present disclosure.

The example embodiment shown in FIG. 2 is configured to minimize electromechanical components in the fluidic disposable module 12 for cost optimization, as well as to achieve a more environmentally sustainable configuration by reducing environmental weight of the disposed part, and to allow use of other sterilization techniques than Ethylene oxide that is the conventional method to sterilize electronic systems. The example embodiment described in FIG. 2 of the present disclosure illustrates a global architecture having a disposable fluidic module and a reusable electromechanical module, as well as a pressure measurement system for patch pumps that is described in co-pending application U.S. application Ser. No. 18/884,259, filed Sep. 13, 2024 entitled “Wearable Fluid Delivery System With Pressure Measurement System Externalized From Fluid Path Between Reservoir And Outlet To Patient” (Applicant ref. P-28782.US01) and incorporated herein by reference in its entirety. The pressure measurement system described herein can be useful for large volume delivery systems (e.g., a patch pump that can deliver on the order of 10 to 50 mL of fluid medicament), as well as for smaller volume delivery systems (e.g., a patch pump that can deliver on the order of 3-9 mL of fluid medicament).

FIG. 2 depicts an example embodiment wherein the wearable FDD has releasably connected parts 102 and 106 of the housing 100 that each contain respectively a disposable fluidic module 12 and an electromechanical module 14, whereby the electromechanical module 14 can be reusable. The releasably connected parts 102 and 106 of the housing 100 have a mechanical interface indicated generally at 108a. For example, the releasably connected parts 102 and 106 can each have hooks or other housing members that are press-fit together or otherwise frictionally engaged to retain the parts 102 and 106 of the housing 100 together (i.e., until a user intentionally separates the housing parts 102 and 106 from each other).

With continued reference to the example embodiment shown in FIG. 2, an electronic/electrical interface 112a is provided between the control electronics 26 and the motor 28 in the electromechanical module 14. The motor 28 in the electromechanical module 14 is at least mechanically connected to the pump 22 as indicated at 108b. For example, a component of the motor 22 such as a rotatable shaft, or rollers in a peristaltic pump, can be releasably mechanically coupled to a pump component (e.g., a pump manifold that receives a rotatable shaft, or the exterior of a fluidic tube in the fluid path 16) for motor-pump engagement. The electromechanical module 14 has another mechanical interface 108c between the pump 22 in the electromechanical module 14 that is disposed in the housing part 106, and the fluid path 16 in the fluidic module 12 that is disposed in the housing part 102. For example, at least part of the fluid path 16 can comprise a compressible material that encases the fluid such as a hose or tubing 16 that can be selectively compressed by a pump 22 configured as a peristaltic pump with rollers and pinions that contact the exterior of the fluid path tubing 16. Different example peristaltic pumps are described below in accordance with various embodiments of the present disclosure. The housing part 102 can comprise an opening wherein rollers or other tubing compressing component of the peristaltic pump 22 can externally and controllably contact the tubing 16 to selectively control movement of fluid in the fluid path 16 from the reservoir 18 to the outlet port 20.

With continued reference to the example embodiment shown in FIG. 2, an optional mechanical interface 108d can be provided between the pressure sensor 24 in the electromechanical module 14 and the fluid path 16 (e.g., tubing or other encapsulated fluid channel configuration) in the fluidic module 12. The pressure sensor 24 is integral to, or separate from but electrically connected to, the control electronics 26 in the electromechanical module 14 as indicated at 112b. The pressure sensor 24 interface 108d to the fluid path 16, and the pump 22 interface 108c to fluid path 16, can both be indirect. For example, the pump 22 can be a peristaltic pump that has indirect contact with tubing in the fluid path 16 (e.g., the housings 102 and 106 have respective and at least partially aligned openings through which the pump mechanisms can access the tubing). The pressure sensor 24 can be configured to cooperate with a membrane that is part of the fluid path 16 in the fluidic module 12 and therefore be in indirect contact with the fluid path 16 fluidic module 12. Examples of such pressure sensors 24 cooperating with a membrane in the fluid path 16 are described in the afore-mentioned co-pending U.S. application Ser. No. 18/884,259, filed Sep. 13, 2024.

The example embodiment shown in FIG. 2 has a fluidic module 12 that is a disposable, single use system without any electronic parts, which can be beneficial after its disposal in terms of refuse sorting at a waste management center. The electromechanical module 14 is a reusable, multiple use system by a single patient and/or multiple patients. Pressure in the fluid path 16 can be optionally measured (e.g., for detecting occlusion or other malfunction of the FDD 10) from an electronic part (e.g., a pressure sensor 24 operating with control electronics 26) in the electromechanical module 14, and can be connected to the fluid path 16 in the disposable fluidic module 12 while remaining outside of the fluid path 16.

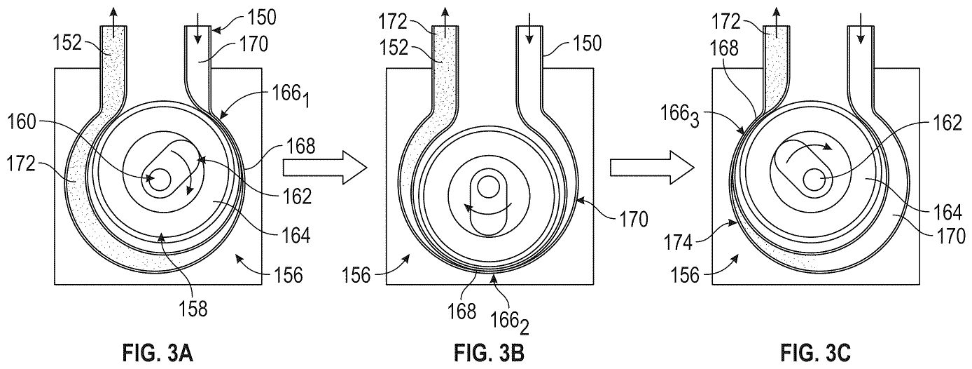

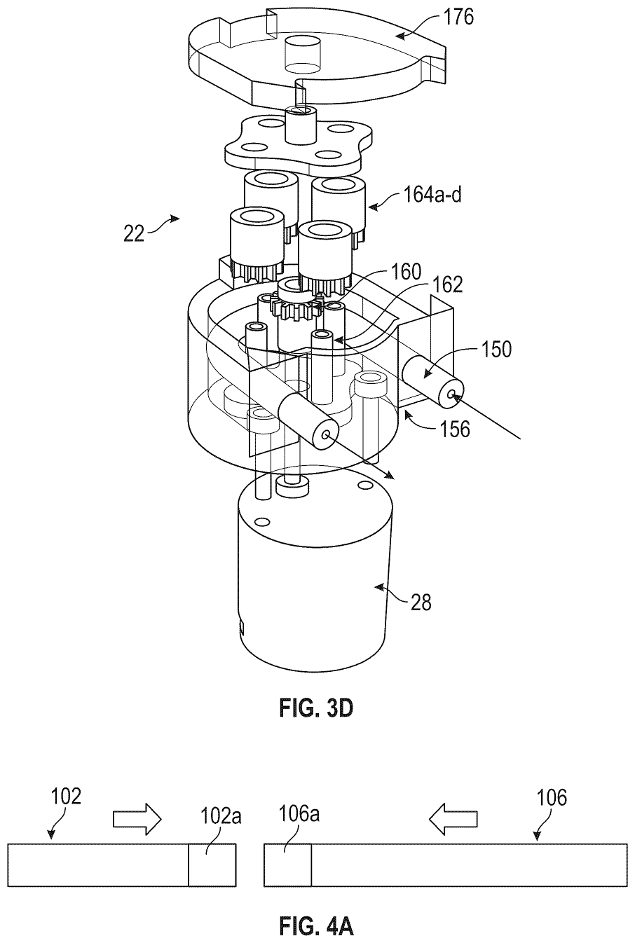

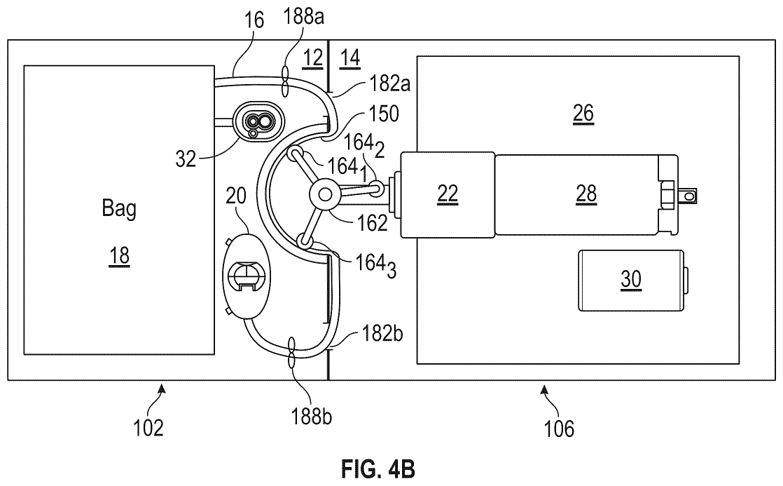

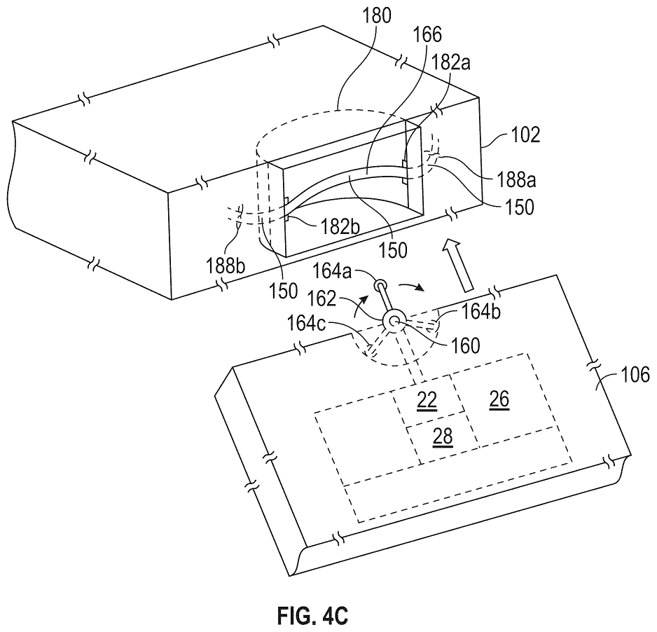

Different example divisions of FDD 10 components among the fluidic and electromechanical modules 12 and 14 are described in the afore-mentioned co-pending U.S. application Ser. No. 18/884,259, filed Sep. 13, 2024 to accommodate the ability to separate the respective housings 102 and 104 in which these modules 12 and 14 are disposed, and to achieve one or more advantages. For example, electronics such as the control electronics 26 and the motor 28 and the power storage system 30 can be isolated to the electromechanical module 14 for sorting separately, when disposed, from the fluidic module 12 that has primarily plastic parts. While the pump can be placed in the fluidic module 12, the pump 22 can also be placed in the electromechanical module 14 as illustrated in FIG. 2 of the present disclosure wherein the electromechanical module 14 is reusable to maximize reusable parts of the FDD 10. Configuring the FDD 10 in FIG. 2 to separate a fluidics module 12 from an electromechanical module 14 avoids or at least minimizes electronics in the fluidics module 12. Also, the example embodiment of the FDD 10 in FIG. 2 externalizes an optional pressure sensor 24 (e.g., a fluid path 16 detection area is accessible from outside of the fluidics module 12) to allow detection of pressure differential such as detection of an occlusion between the reservoir 18 and the outlet port 20 in the fluidics module 12 from a pressure sensor 24 disposed in the electromechanical module 14. In addition, the embodiment of FDD 10 in FIG. 2 of the present disclosure allows for more options for sterilization. For example, certain sterilization methods such as gamma or X-ray radiation methods can be used to sterilize the fluidics module 12 when there are no electronics within the fluidics module 12 that can be harmed by gamma or X-ray radiation. A FDD 10 is more sustainable when it uses only mechanical parts in the fluidics module 12 (e.g., the FDD 10 in FIG. 3 or 4) and gamma or X-ray sterilization thereof. The reusable electromechanical module 14 can be packaged separately as cartridges and does not need to be sterilized at all. In addition, the more components of the FDD 10 that are disposed in the reusable electromechanical module 14, the more optimized that FDD 10 is for minimizing direct costs of producing the FDD 10 (i.e., Cost of Goods Sold (COGS)).

It is to be understood that FDD 10 is generally for bolus or infusion and can use various delivery modes (e.g., pulsed or continuous delivery), and can employ any of these various modes for delivery over a relatively short period of time, or over longer periods of time. Further, the FDD 10 can be employed to deliver various medicaments (e.g., various drugs and/or other various medical fluids) for various medical fluid delivery applications. Example pumps 22 are described herein, but it should be understood that other types of pumps can be used and various delivery methods can be used by the example embodiments such as, but not limited to, the above-reference delivery modes.

The pump 22 in the electromechanical module 14 is considered as a peristaltic pump; that is, a rotary or linear peristaltic-type, positive displacement pump that can move fluids of many viscosities through a tight, flexible and compressible fluid path or tubing or hose 16a used for at least a portion of the fluid path 16 that fluidically connects the fluid reservoir 18 in the fluidic module 14 to the outlet port 20 to the patient. The pump 22 can therefore transport fluid from the reservoir 18 without the fluid coming into contact with the pump 22 components or other components of the electromechanical module 14. As such, the pump 22 and other components of the electromechanical module 14 do not have to be tested for chemical compatibility with the fluid being delivered from the fluid reservoir 18. Different configurations of a peristaltic pump can be employed for the pump 22, and a few different examples of a peristaltic-type pump 22 are described herein in accordance with illustrative embodiments of the present disclosure. These different examples of a peristaltic-type pump 22 have some common features such as a pump mechanism that contacts the fluid path 16 tubing and comprises rollers or other drive means to selectively compress the fluid path 16 tubing along one or more points along the length of the tubing.

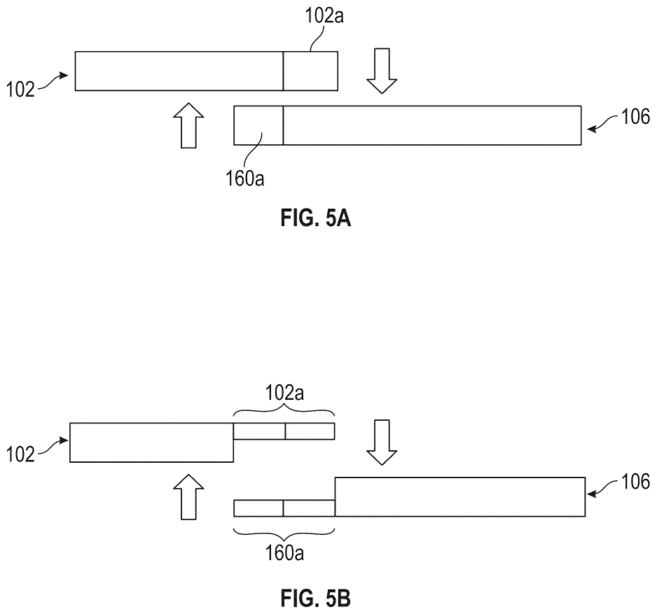

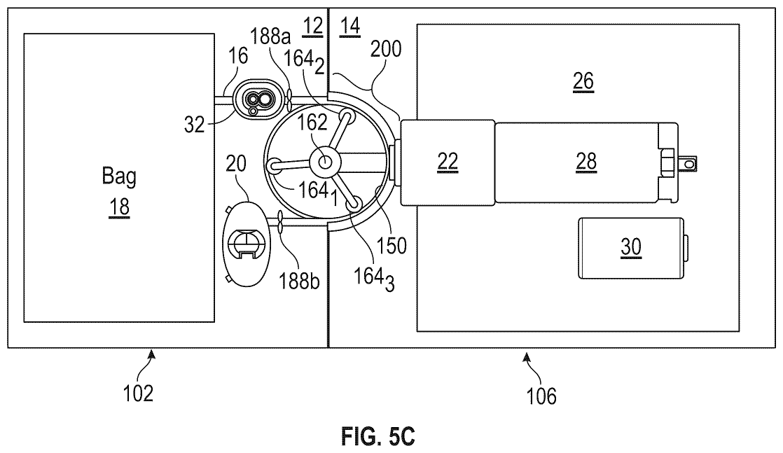

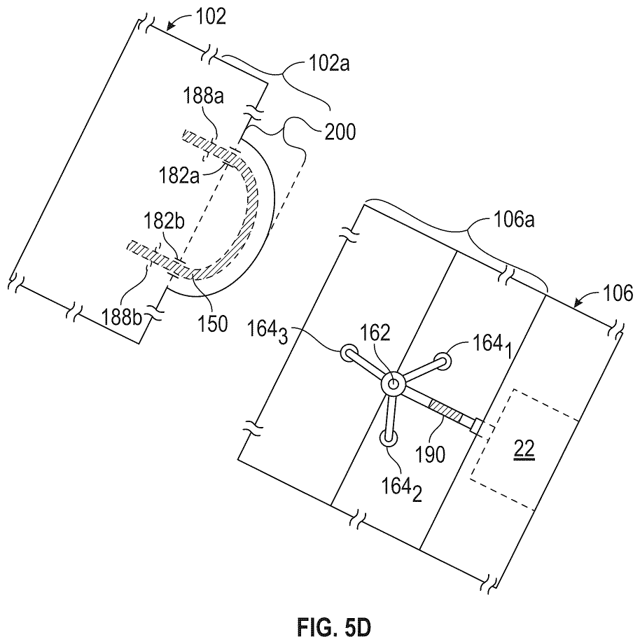

FIGS. 3A, 3B and 3C depict top views of operations of a peristaltic-type pump that can be deployed as the pump 22 in an electromechanical module 14, which is configured to be releasably coupled to a fluidic module 12 in accordance with an aspect of an example embodiment of the present disclosure. A portion of the fluid path 16 in the fluidic module 12 comprises compressible tubing 16a represented, for example, at 150 in FIGS. 3A-6F below. As described below, the tubing 150 can be provided in a base or housing 156. The base or housing 156 can correspond to a portion of the housing 102 of the fluidic module 12, or correspond to a portion of the housing 106 of the electromechanical module 14. For example, a pump head 158 is provided that comprises a rotating shaft 160 having an eccentric rotor 162 and ring 164 assembly connected thereto as shown in FIG. 3C, or can have a rotor 162 and respective roller(s) 164₁₋ₙ connected thereto as shown in FIG. 3D. FIG. 3D is an exploded view of another example peristaltic-type pump that can be deployed as the pump 22 in an electromechanical module 14 wherein the rotor 162 operates with a pinion 160 and roller(s) 164₁₋ₙ to apply pressure at one or more contact points 166₁₋ₙ along the tubing 150. FIG. 3D illustrates a vertical arrangement of the pump motor 28 with respect to the pinion 160 and rotor 162. It is to be understood that the motor 28 can be arranged laterally with respect to the rotating shaft or pinion 160 and the eccentric rotor 162 and ring 164 assembly or roller(s) 164₁₋ₙ (e.g., as illustrated in the example embodiments shown in FIGS. 4B and 5C) using conventional motor mounting hardware and gear assembly to minimize the thickness of the electromechanical module 14 and therefore the wearable fluid delivery device 10.

The pump head 158 is disposed proximally to the tubing 150 such that its eccentric rotor 162 and ring 164 assembly per FIG. 3C, or rotor 162 with roller(s) 164₁₋ₙ per FIG. 3D, compress the tubing 150 at one or more contact points 166₁₋ₙ along the length of the tubing 150, depending on the angular arrangement of the eccentric rotor 162, or angular arrangement of the roller(s) 164₁₋ₙ around the circumference of the pump head 158, whichever is used. When the eccentric rotor 162 and ring 164 assembly per FIG. 3C, or rotor 162 and roller(s) 164₁₋ₙ per FIG. 3D, in the peristaltic-type pump compress the tubing 150 as they rotate, a vacuum is created that draws fluid through the tubing 150, as indicated in FIGS. 3A and 3B. When the part 168 of the tubing 150 under compression is closed, fluid is forced to move through the tubing 150 as indicated at 172. Additionally, as a compressed, closed tubing section opens to its natural decompressed state after the eccentric rotor 162 and ring 164 assembly per FIG. 3C or roller(s) 164₁₋ₙ per FIG. 3D pass, more fluid is drawn into the tubing 150 as illustrated at 174 in FIG. 3C.

Different example embodiments of releasably connected housing parts 102 and 106 containing a fluidic module 12 and an electromechanical module 14, respectively, that allow a peristaltic-type pump 22 deployed in the electromechanical module 14 to controllably contact a section of the fluid path in the fluidic module 12 are described herein with respect to FIGS. 4A-4C, FIGS. 5A-5D, and FIGS. 6A-6F. It is to be understood that the relative dimensions of the housing parts 102 and 106 and their corresponding components shown in FIGS. 4A-4C, FIGS. 5A-5D, and FIGS. 6A-6F are for illustrative purposes and may not necessarily reflect actual dimensions.

In accordance with an example embodiment, the base or housing 156 in FIGS. 3A-3C that supports a section of the fluid path 16 (i.e., tubing 150), which is to be controllably compressed by a peristaltic-type pump 22 in an electromechanical module 14, is implemented as a portion of the housing 102 of the fluidic module 12. As will be described with reference to FIGS. 4A, 4B and 4C, the fluid delivery device 10 can be configured to have releasably connected housing parts 102 and 106 containing the fluidic module 12 and the electromechanical module 14, respectively, that are engaged side-by-side. As illustrated in FIGS. 4A and 4B, each housing part 102, 106 has at least a portion thereof 102a, 106a that overlaps with the other housing part's overlapping portion 106a, 102a, respectively, when the housing parts 102 and 106 are engaged (e.g., releasably coupled together). FIG. 4B is a top view of the housing parts 102 and 106 engaged together and with the tops of the housing parts 102 and 106 removed for clarity. A fluid path 16 is shown extending from a fill port 32 to a reservoir 18 and then to an outlet port 20. At least part of the fluid path 16 can comprise tubing 150. For example, the fluid path 16 can comprise different types of fluid conduits (e.g., embedded channels in a baseplate of the housing 102) including sections of tubing 150 that are fluidically connected to each other and sealed to prevent leakage of fluid using one or more conventional fluidic connectors and/or methods for achieving same. Alternatively, the fluid path 16 can be entirely made of a compressible fluid conduit material that can cooperate with peristaltic operations of a pump 22 to controllably fill or otherwise draw fluid from the reservoir 18 in the fluid path 16 and to controllably transport the fluid in the fluid path at a controlled volume and rate to the outlet port 20.

The housing part 102 shown in FIGS. 4B and 4C comprises a recess 180 in a side wall thereof and at least one fluid path access opening to allow a section of the fluid path tubing 150 to be exposed externally with respect to the housing 102. In the illustrated embodiment, two fluid path access openings 182a and 182b are shown in or proximal to the recess 180 with a section of fluid path tubing 150 that exits an interior of the housing 102 from path access opening 182a, extends along a lateral dimension of the recess 180, and then re-enters the interior of the housing 102 via path access opening 182b. The housing 102 can comprise non-compressive clamps or other mounting hardware indicated at 188a,b along one or more points along the fluid path 16 to secure the tubing 150 within the housing part 102 (e.g., secure the tubing 150 to a side wall or baseplate or other component in the housing part 102). The recess 180 can constitute the overlapping portion 102a of the housing 102 and is dimensioned receive an overlapping portion 106a of the housing 106 when the housings 102 and 106 are releasably engaged. The overlapping portion 106a of the housing 106 comprises at least one or more of a pump head contact member 164 of the pump 22. When engaged, the motor 18 of the pump 22 is operated by the control electronics 26 to cause the pump head contact member(s) 164 of the pump 22 (e.g., an eccentric rotor 162 and ring 164 assembly as shown in FIG. 3C, or the respective roller(s) 164₁₋ₙ shown in FIG. 3D) to rotate and controllably contact the tubing 150 to achieve a peristaltic operation (e.g., to draw fluid into the tubing 150, or expel fluid from the tubing 150, as described above in connection with FIGS. 3A-C).

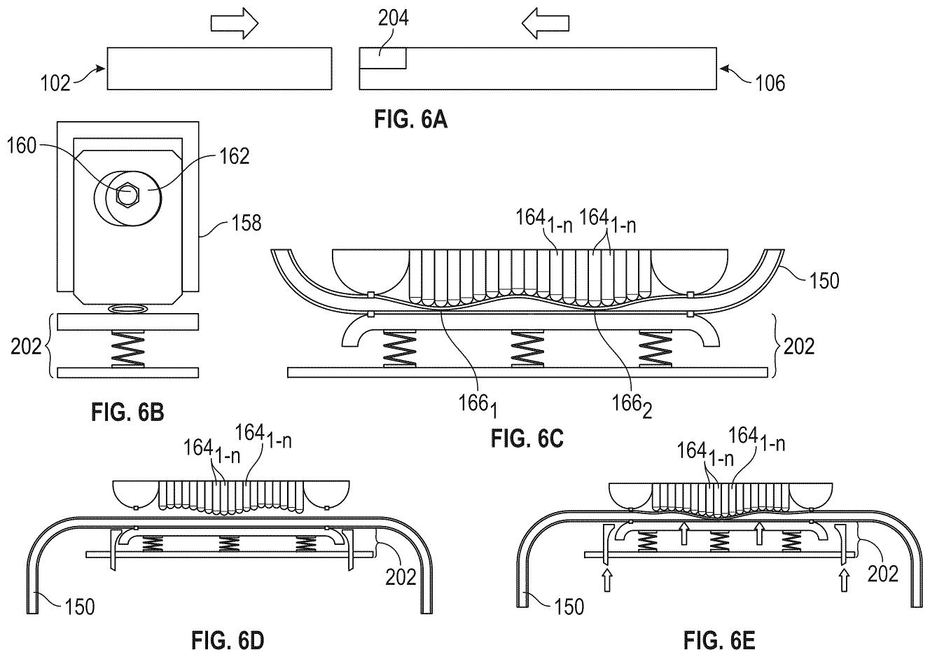

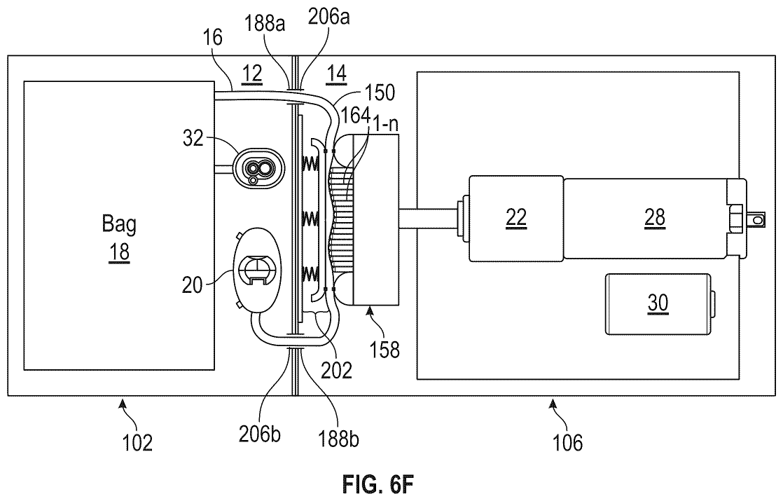

The electromechanical module 14 in the housing part 106 can be provided with a retraction assembly connected to the pump 22 that is operable to selectively laterally move the pump head 158 (e.g., a pump head comprising rotating pinion 160, rotor 162 and rollers 164₁₋ₙ) closer to or farther from the tubing 150 in the recess 180 to avoid pinching the tubing 150. For example, the optional pressure sensor 24 or other sensor (e.g., a sensor deployed proximal to the pump 22 that can detect how much tension the pump head 158 is subjected to when the housing parts 102,106 are engaged and the pump head contact member(s) 164 are contacting the tubing 150) can provide sensor data to the control electronics 26 that, in turn, operate a telescopic piece of the pump 22 axis indicated at 190 in FIG. 5D to selectively extend or retract it along the longitudinal axis of the pump 22 to change the pressure applied by the pump head contact member(s) 164 to the tubing 150). This telescopic piece or arm 190 can also be used to tune the pressure on the tubing to adapt to counter pressure generated by injection in tissues. For example, if pressure increases in a downstream fluid path, the pump can be configured 22 can be configured to increase tightness at the tubing level (e.g., via the degree of extension of a retractable member such as the telescopic piece or arm 190) and, if pressure is low, it can be sufficient to fully compress the tubing without overpressure in tubing via the telescopic piece 190. It is to be understood that the retractable member 190 can be implemented using different components than those shown in FIG. 5D and can be provided to the pumps shown in the embodiments illustrated in FIGS. 4A-4C and FIGS. 6A-6F, as well as the embodiment shown in FIGS. 5A-5D.

With regard to the embodiment shown in FIGS. 5A-5D, the base or housing 156 in FIGS. 3A-3C that supports a section of the fluid path 16 (i.e., tubing 150), which is to be controllably compressed by a peristaltic-type pump 22 in an electromechanical module 14, is implemented as a portion of the housing 102 of the fluidic module 12. As will be described with reference to FIGS. 5A-5D, the fluid delivery device 10 can be configured to have releasably connected housing parts 102 and 106 containing the fluidic module 12 and the electromechanical module 14, respectively, that are engaged using a stacked or vertical arrangement of portions 102a, 106a thereof. As illustrated in FIGS. 5A and 5B, each housing part 102, 106 has at least a portion thereof 102a, 106a that overlaps with the other housing part's overlapping portion 106a, 102a, respectively, when the housing parts 102 and 106 are engaged (e.g., releasably coupled together). FIG. 5C is a top view of the housing parts 102 and 106 engaged together and with the tops of the housing parts 102 and 106 removed for clarity. A fluid path 16 is shown extending from a fill port 32 to a reservoir 18 and then to an outlet port 20. As stated above on connection with FIGS. 4A-4C, at least part of the fluid path 16 can comprise tubing 150. For example, the fluid path 16 can comprise different types of fluid conduits (e.g., embedded channels in a baseplate of the housing 102) including sections of tubing 150 that are fluidically connected to each other and sealed to prevent leakage of fluid using one or more conventional fluidic connectors and/or methods for achieving same. Alternatively, the fluid path 16 can be entirely made of a compressible fluid conduit material that can cooperate with peristaltic operations of a pump 22 to controllably fill or otherwise draw fluid from the reservoir 18 in the fluid path 16 and to controllably transport the fluid in the fluid path at a controlled volume and rate to the outlet port 20.

With continued reference to FIGS. 5A and 5B, each housing part 102, 106 has at least a portion thereof 102a, 106a that overlaps with the other housing part's overlapping portion 106a, 102a, respectively, when the housing parts 102 and 106 are engaged (e.g., releasably coupled together). The overlapping portions 102a, 106a can have less thickness than the rest of the corresponding housing part 102 and 106, as shown in FIG. 5B, such that when the housing parts 102 and 106 are engaged, the coupled, vertically stacked overlapping portions 102a, 106a have essentially the same thickness as the remainders of the housing parts 102 and 106. The overlapping portion 102a of the housing part 102 can include a section of the tubing 150 that is exposed.

The housing part 102 shown in FIGS. 5C and 5D comprises a side wall extension 200 in the overlapping portion 102a wherein at least part of the fluid path tubing 150 is exposed externally with respect to the rest of the housing 102. The remainder of the housing part 102 can be enclosed and have fluid path access openings 182a and 182b that allow a section of fluid path tubing 150 to exit an interior of the housing part 102 from path access opening 182a, extend along a lateral dimension of the side wall extension 200, and then re-enter the interior of the housing 102 via the fluid path access opening 182b. The overlapping portion 106a of the housing part 106 has the pump head 158 mounted thereon with at least one pump head contact member 164 exposed when the housing parts 102 and 106 are disengaged from each other. The tubing 150 extends along the inner wall of the side wall extension 200. The side wall extension 200 is dimensioned accommodate at least a portion of the angular path arrangement of the at least one pump head contact member 164 to selectively compress the tubing 150 for a peristaltic pump operation. For example, when the housing parts 102 and 106 are stacked and engaged, the motor 18 of the pump 22 is operated by the control electronics 26 to cause the pump head contact member(s) 164 of the pump 22 (e.g., an eccentric rotor 162 and ring 164 assembly as shown in FIG. 3C, or the respective roller(s) 164₁₋ₙ shown in FIG. 3D) to rotate along the angular path of the pump head 158 components 164 and controllably contact the tubing 150 in the side wall extension 200 to achieve a peristaltic operation (e.g., to draw fluid into the tubing 150, or expel fluid from the tubing 150, as described above in connection with FIGS. 3A-C). As stated above, the pump 22 can be provided with a retractable member 190 to control the pressure of the at least one pump head contact member 164 against the tubing 150 when the housing parts 102 and 106 are engaged to each other.

As illustrated in FIG. 6A, the example embodiment shown in FIGS. 6A-6F comprises housing parts 102, 106 that do not need overlapping portions 102a, 106a, unlike the embodiments of FIGS. 4A-4C and 5A-5D. The pump 22 in the example embodiment shown in FIGS. 6A-6F is a linear peristaltic pump. With reference to FIGS. 6B and 6C, which are partial side and front cross-sectional views of an example linear peristatic pump head 158, the motor 18 of the pump 22 rotates a crankshaft 160 that cooperates with eccentric discs indicated at 162 to selectively displace vertically movable actuators 164₁₋ₙ along a spring-loaded plate 202 to create 166₁₋ₙ contact points on tubing 150 provided on the spring-loaded plate 202. The pump head 158 assembly is provided within the housing part 106 which can have a lid 204 to expose at least part of the interior of the housing part 106 to allow access to the spring-loaded plate 202 and placement of tubing 150 exposed outside the housing part 102 onto the plate 202 when the housing parts 102 and 106 are engaged. The housing part 106 has openings 206a,b to receive a section of the tubing 150 therein. To expose a section of the tubing 150, the housing part 102 has fluid path access openings 182a and 182b that allow a section of fluid path tubing 150 to exit an interior of the housing part 102 from path access opening 182a, extend along a lateral dimension of the side wall extension 200, and then re-enter the interior of the housing 102 via the fluid path access opening 182b.

FIG. 6F is a top view of the housing parts 102 and 106 engaged together and with the tops of the housing parts 102 and 106 removed for clarity. With reference to FIGS. 6D and 6E, a user can remove the lid 204 from the housing 106 to expose the spring-loaded plate 202, compress the plate 202 and place the tubing 150 thereon, as shown in FIG. 6D. The plate 202 can then be released to allow the vertically movable actuators 164 to contact the tubing 150 at various contact points 166₁₋ₙ, depending on how the control electronics 26 actuate the pump 22 for peristaltic operations.

As stated above, by moving the pump 22 into an electromechanical module 14, the electromechanical module 14 can be configured to be reusable with replacement and disposal of the fluidic module 12 only, which would make the FDD 10 much more environmentally sustainable. Such a configuration for an FDD 10 also realizes advantages in terms of expanded options for means of sterilization such as relying less on ethylene oxide (EtO) sterilization and using instead Gamma sterilization, for example, which is incompatible with the electronic components found in some wearable FDD configurations.

In accordance with another aspect of the example embodiments of the present disclosure, artificial intelligence (AI) can be integrated into the control system of an FDD 10. For example, fluid flow injection can be implemented depending on the state of the patient as monitored through sensors integrated into the electromechanical module 14 (e.g., temperature, skin pH, skin tension, and so on). AI can be particularly useful due to the multiplicity of patient parameters to monitor. For example, by reusing the electromechanical system 14, the FDD 10 can be trained differently from one patient to the next, depending that patient's reaction to the medical treatment as determined by the sensors and processing of sensor outputs.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. Also, the drawings are representative of example components in the illustrative embodiments and these representations can have dimensional scale different from that shown in the FIGS. 1-11.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., erasable programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A wearable fluid delivery device comprising:
a first housing that defines an interior comprising a fluidics module and that has a fluid path access opening to an exterior of the first housing, the fluidics module comprising a reservoir of fluid and a fluid path that fluidically connects the reservoir to an outlet port configured to output the fluid from the fluid path to a patient, the fluid path comprising a compressible section thereof that extends from the interior of the first housing through the fluid path access opening to an exterior of the first housing; and
a second housing separate from the first housing and configured to be releasably connected to the first housing and operated as the wearable fluid delivery device whereby the connected first housing and second housing are dimensioned for detachable application at an injection site on a patient's skin;
the second housing comprising an electromechanical module and a pump access opening, the electromechanical module comprising a pump connected to a motor and electronics configured to operate the motor, the fluid path access opening and the pump access opening being at least partially aligned when the second housing is connected to first housing, the pump comprising a rotor connected to at least one pump head contact member disposed at the pump access opening of the second housing and the motor controlled by the electronics to selectively contact the compressible section of the fluid path with the at least one pump head contact member for peristaltic pumping operations chosen from filling the fluid path with fluid from the reservoir and transporting the fluid in the fluid path toward the outlet port.

2. The wearable fluid delivery device of claim 1, wherein the first housing is disposable after a first use thereof and replaceable by new first housing that is similar to the used first housing, and the second housing is reusable by disconnecting the used first housing from the second housing and attaching the new first housing to the second housing to engage the at least one pump head contact member with the compressible section of the fluid path in the new first housing.

3. The wearable fluid delivery device of claim 1, wherein at least one of the connected first housing and second housing comprises an adhesive patch to attach the connected first housing and second housing to the patient's skin.

4. The wearable fluid delivery device of claim 1, wherein the compressible section of the fluid path is chosen from tubing and hose.

5. The wearable fluid delivery device of claim 1, wherein each of the first housing and the second housing has a main portion that is characterized by a respective main housing thickness, and the connected first housing and second housing assembly has a connected housing thickness that is the same as the main housing thickness.

6. The wearable fluid delivery device of claim 5, wherein the first housing has an overlapping portion that comprises the compressible section of the fluid path and that is combined with an overlapping portion of the second housing to connect the pump of the second housing to the compressible section of the fluid path.

7. The wearable fluid delivery device of claim 6, wherein a section thickness of the combined overlapping portions does not exceed the main housing thickness.

8. The wearable fluid delivery device of claim 6, wherein the overlapping portion of the first housing is moved laterally with respect to the overlapping portion of the second housing to connect the first housing to the second housing, the overlapping portion of the first housing having at least part of the compressible section of the fluid path exposed along a side wall of the first housing and disposed between the side wall of the first housing and an adjacent side wall of the second housing.

9. The wearable fluid delivery device of claim 8, wherein the side wall of the first housing comprises a recess with a fluid path access opening arranged therein to expose the at least part compressible section of the fluid path.

10. The wearable fluid delivery device of claim 8, wherein the at least one pump head contact member extends from the second housing to contact the exposed compressible section of the fluid path within the recess when the first housing and the second housing are connected.

11. The wearable fluid delivery device of claim 6, wherein the overlapping portion of the first housing is moved in a vertically stacked arrangement with respect to the overlapping section of the second housing to connect the first housing to the second housing.

12. The wearable fluid delivery device of claim 11, wherein the overlapping portion of the first housing is a side wall extension with exposed fluid path tubing arranged along an interior side wall thereof, the side wall extension being dimensioned for placement over a pump head of the pump having at least one pump head contact member in the overlapping portion of the second housing, the side wall extension and the overlapping portion of the second housing being arranged together such that the at least one pump head contact member controllably compresses the tubing against the interior side wall.

13. The wearable fluid delivery device of claim 1, wherein the pump is a linear peristaltic pump comprising a spring loaded plate arranged within the second housing, and the compressible section of the fluid path that is exposed at the exterior of the first housing is arranged along the spring loaded plate when the first and second housing are engaged.

14. The wearable fluid delivery device of claim 13, wherein the second housing comprises a fluid path access opening from an exterior of the second housing to an interior thereof to receive the compressible section of the fluid path for arrangement along the spring loaded plate.

15. The wearable fluid delivery device of claim 1, further comprising a retractable member for the pump that controllably extends and retracts the pump head contact member with respect to the compressible section of the fluid path.
